(19) United States
(12) Patent Application Publication
(10) Pub. No.: US 2011/0208057 A1
(43) Pub. Date: Aug. 25, 2011

(54) SUBJECT INFORMATION PROCESSING APPARATUS

(75) Inventor: Katsuya Oikawa, Tokyo (JP)

(73) Assignee: CANON KABUSHIKI KAISHA, Tokyo (JP)

(21) Appl. No.: 13/029,245

(22) Filed: Feb. 17, 2011

(30) Foreign Application Priority Data
Feb. 24, 2010 (JP) 2010-038977

(51) Int. Cl. A61B 8/14

(52) U.S. Cl. 600/443

(57) ABSTRACT

A subject information processing apparatus having a first device array for transmitting/receiving an elastic wave; a first signal processor for generating a tomographic image from a signal received by the first device array; a second device array for receiving an elastic wave which is generated by irradiating light onto a subject; and a second signal processor for generating a three-dimensional image from a signal received by the second device array. The first device array transmits/receives the elastic wave diagonally with respect to a surface of the subject so that a region in the subject where the tomographic image is obtained and a region in the subject where the three-dimensional image is obtained overlap.

### BACKGROUND OF THE INVENTION

1. Field of the Invention

The present invention relates to a subject information processing apparatus, and more particularly to a subject information processing apparatus which combines a three-dimensional image based on photoacoustic waves and a two-dimensional tomographic image based on ultrasonic echoes.

2. Description of the Related Art

Image diagnostic apparatuses using ultrasonic waves have been widely used. In the case of conventional apparatuses, a tomographic image is generated by transmitting ultrasonic waves to a subject and receiving and imaging the reflected ultrasonic echoes. Three-dimensional images can also be obtained by using two-dimensionally arrayed electro-mechanical transforming devices (transducers) or scanning one-dimensionally arrayed transducers. It has also been proposed to display such a three-dimensional image along with a two-dimensional tomographic image (Japanese Patent Application Laid-Open No. 2008-229097).

In subject inspection, on the other hand, the development of apparatuses which display not only shape images but also functional images is now ongoing. One such apparatus is an apparatus utilizing a photoacoustic spectral analysis method. The photoacoustic spectral analysis method detects photoacoustic waves which are generated by a specific substance in the subject, absorbing the energy of light having a predetermined wavelength in visible, near infrared or intermediate infrared light, which is irradiated onto the subject, and measures the density of the specific substance quantitatively. A specific substance in the subject is, for example, glucose or hemoglobin contained in blood.

According to Japanese Patent Application Laid-Open No. 2005-21380, both a photoacoustic image and normal ultrasonic echo image are simultaneously obtained by a common one-dimensional transducer, whereby the shape image and functional image are displayed. It is expected that the malignant tumor in a tissue can be effectively determined by displaying while superposing the structure of the tissue, obtained by the ultrasonic echo method, onto the three-dimensional distribution structure of glucose and hemoglobin and the activity thereof obtained by the photoacoustic imaging method. Particularly in the case of the functional image generated by the photoacoustic imaging method, only the area having a specific function is displayed, so visibility in the three-dimensional display is good, but determining the location in the body is difficult. The ultrasonic echo method, with which general structure of the tissue is displayed, is advantageous in specifying the location, and it is effective to display this image together with the photoacoustic imaging.

In this description, an elastic wave generated by the photoacoustic spectral analysis method (photoacoustic imaging method) is called “photoacoustic wave”, and an elastic wave transmitted/received by a normal pulse echo method is called an “ultrasonic wave”.

In the case of Japanese Patent Application Laid-Open No. 2008-229097 which discloses that a two-dimensional tomographic image generated by the ultrasonic echo method and a three-dimensional image are simultaneously displayed, a functional image cannot be obtained. An advantage of the ultrasonic echo method is that an image of soft tissue can be captured in detail, but visibility drops if this method is used for a three-dimensional image. The three-dimensional image generated by the ultrasonic echo method is applied to observe the area having clear boundaries, such as a heart and fetus, and if it is used for observing a plurality of tissues of which boundaries are not clear, the plurality of areas overlap by creating a three-dimensional image, which drops visibility. Furthermore in order to obtain a three-dimensional image using the ultrasonic echo method, many ultrasonic beams must be sequentially created and an ultrasonic echo must be obtained for each of the ultrasonic beams. This means that it is difficult to create a detailed image having high resolution in a short time.

In the case of obtaining a three-dimensional image using the photoacoustic imaging method, on the other hand, the three-dimensional image data can be constructed by receiving the photoacoustic waves generated by one light irradiation using two-dimensionally arrayed transducers. Therefore if the method disclosed in Japanese Patent Application Laid-Open No. 2005-21380 is used, time for obtaining data does not increase, unlike the case of obtaining three-dimensional image data generated by the ultrasonic echo method.

However according to Japanese Patent Application Laid-Open No. 2005-21380, a common transducer is used for receiving a photoacoustic wave, transmitting an ultrasonic beam, and receiving the echo thereof, so the following problems are generated.

A frequency band of a photoacoustic wave used for a photoacoustic spectral analysis method is generally lower than the frequency band of ultrasonic waves used for the ultrasonic echo. For example, the frequency band of the photoacoustic wave is in the 200 KHz to 2 MHz range with 1 MHz as a central frequency, which is lower than the center frequency of 3.5 MHz to 12 MHz which is used for the ultrasonic echo. Therefore if both of these waves are received by a common transducer, the spatial resolution deteriorates in the ultrasonic image. Japanese Patent Application Laid-Open No. 2005-21380 uses a harmonic imaging method to deal with this problem, but harmonic components make signals attenuate more than fundamental components, so sensitivity may drop. If the frequency bands of the photoacoustic wave and ultrasonic wave are more distant (e.g. central band of photoacoustic wave is about 1 MHz, and the central band of the ultrasonic wave is about 10 MHz), this problem becomes more conspicuous if a common transducer is used for reception.

As mentioned above, in order to construct a three-dimensional image at high-speed using the photoacoustic imaging method, two-dimensional transducers are required. In order to obtain image data in a short time using the ultrasonic echo method, on the other hand, it is preferable to construct a tomographic image by performing ultrasonic beam scanning on the plane using transducers, which are arrayed in approximately one dimension.

In this way, the ultrasonic echo method and the photoacoustic imaging method have different demands for transducers, so it is preferable to use independent transducers respectively. In this case, the regions to obtain actual images deviate due to the deviation of the positions of the respective transducers.

### SUMMARY OF THE INVENTION

With the foregoing in view, it is an object of the present invention to match the image capturing regions when a three-dimensional image generated by the photoacoustic imaging method and a two-dimensional tomographic image generated by the ultrasonic echo method are obtained by different transducers.

The present invention in its first aspect provides a subject information processing apparatus having: a first device array for transmitting/receiving an elastic wave; a first signal processor for generating a tomographic image from a signal received by the first device array; a second device array for receiving an elastic wave which is generated by irradiating light onto a subject; and a second signal processor for generating a three-dimensional image from a signal received by the second device array, wherein the first device array transmits/receives the elastic wave diagonally with respect to a surface of the subject so that a region in the subject where the tomographic image is obtained and a region in the subject where the three-dimensional image is obtained overlap.

The present invention in its second aspect provides a subject information processing method for a subject information processing apparatus, the method having: a tomographic image generation step of generating a tomographic image of the subject by irradiating the elastic wave onto the subject, and receiving the echoed elastic wave reflected from inside the subject; and a three-dimensional image generation step of generating a three-dimensional image of the subject by irradiating the light onto the subject, and receiving the elastic wave generated inside the subject, wherein in the tomographic image generation step, the elastic wave is transmitted/received diagonally with respect to a surface of the subject so that a region in the subject where the tomographic image is obtained and a region in the subject where the three-dimensional image is obtained overlap.

According to the present invention, the image capturing regions of a three-dimensional image generated by the photoacoustic spectral analysis method and an ultrasonic tomographic image generated by the ultrasonic echo method overlap, so both images of a same subject area can be obtained at the same time. Furthermore the positional relationship of the inspection target and peripheral biological tissue can be accurately observed in the photoacoustic imaging method, and a region to be imaged by the photoacoustic spectral method can be set while visually checking on the tomographic image of the tissue.

Further features of the present invention will become apparent from the following description of exemplary embodiments with reference to the attached drawings.

### BRIEF DESCRIPTION OF THE DRAWINGS

FIG. 1 is a block diagram depicting a configuration example of a photoacoustic imaging apparatus;

FIG. 2 are diagrams depicting a photoacoustic probe;

FIG. 3 is a diagram depicting a configuration of a probe when the ultrasonic scanning surface is mechanically controlled; and

FIG. 4 is a diagram depicting an example of a photoacoustic imaging apparatus.

### DESCRIPTION OF THE EMBODIMENTS

### First Embodiment

### [General Configuration]

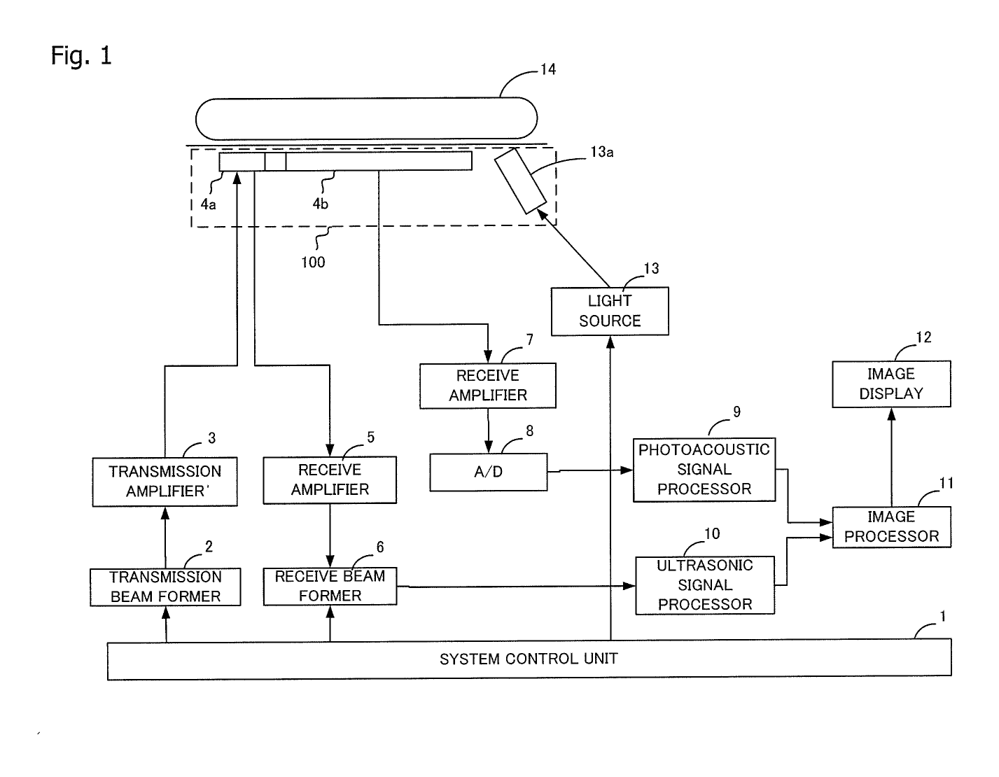

FIG. 1 shows a general configuration of a photoacoustic imaging apparatus (subject information processing apparatus) according to this embodiment. The photoacoustic imaging apparatus according to this embodiment has a photoacoustic probe 100 which further comprises a transducer array 4a for the ultrasonic echo method and a transducer array 4b for the photoacoustic imaging. These transducer arrays are hereafter called an ultrasonic transducer array 4a and a photoacoustic transducer array 4b respectively. The ultrasonic transducer array 4a corresponds to the first device array in the present invention, and the photoacoustic transducer array 4b corresponds to the second device array in the present invention.

First the configuration for generating a tomographic image using the ultrasonic echo method will be described. In order to transmit an ultrasonic wave (elastic wave) from the ultrasonic transducer array 4a, an ultrasonic signal is generated via a system control unit 1, transmission beam former 2 and transmission amplifier 3, and voltage is applied to the ultrasonic transducer array 4a. The transmitted ultrasonic wave is reflected by the subject 14, and the reflected ultrasonic wave (echoed elastic wave) is received by the ultrasonic transducer array 4a. A receive ultrasonic signal in each device is phased and added to the received ultrasonic signal via the receive amplifier 5 and a reception beam former 6 which performs delay and weighting control. The resulting signal is detected and converted into a brightness signal or the like by an ultrasonic signal processing unit (first signal processing unit) 10, and is stored in an image memory in an image processing unit 11.

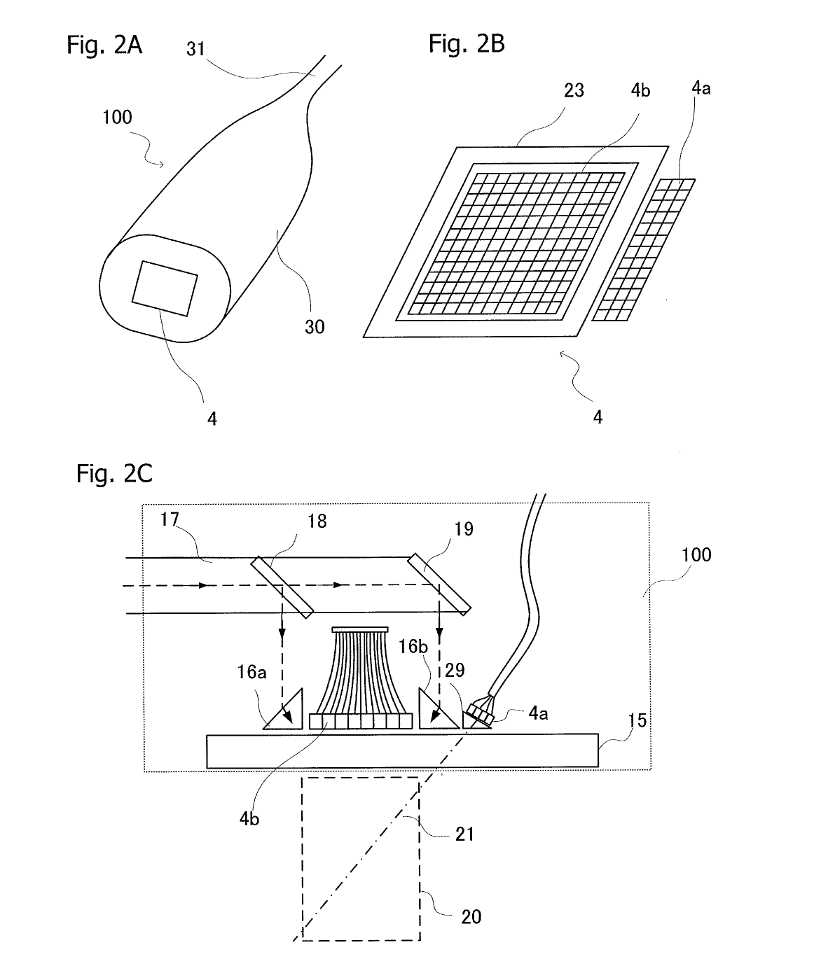

A linear scanning method can be used for the transmission/reception beam forming to create a tomographic image using the ultrasonic echo method. In the linear scanning method, an ultrasonic beam is formed by the ultrasonic transducer array 4a and this beam scans approximately in a parallel direction. For this, a part of the transducer group constituting the ultrasonic transducer array 4a is used as an ultrasonic aperture for transmitting/receiving ultrasonic waves, and an ultrasonic beam is transmitted/received through this ultrasonic aperture portion. The transmission beam former 2 and the reception beam former 6 select the ultrasonic aperture portion, and transmits/receives ultrasonic waves using a corresponding plurality of transducers in the ultrasonic transducer array 4a. By switching the transducers to be selected, the ultrasonic aperture is moved in a one-dimensional direction. In other words, the transmission/reception ultrasonic beam can be moved approximately in a parallel direction. An ultrasonic scanning surface 21 (FIG. 2C) is formed by scanning the ultrasonic beam (linear scanning). The region of capturing the tomographic image using the ultrasonic echo method is this ultrasonic scanning surface 21.

At the same time, the transmission beam former 2 and the reception beam former 6 perform the focusing operation, which converges ultrasonic beams by providing different delays to the transmission/reception signals of a plurality of transducers. It is also desirable to perform dynamic focus for moving a focal point upon phasing and adding of the receive signal, and perform apodization, but a description of these processings, which are widely known in this technical field, is omitted. According to this embodiment, the ultrasonic scanning surface 21 can be tilted by beam forming processing upon transmission/reception, and details of this processing will be described later.

Now a configuration for generating a three-dimensional image using the photoacoustic spectral method will be described. The light source 13 oscillates the pulsed laser beam to be irradiated onto the subject 14 by drive signals from the system control unit 1, and irradiates it onto the subject 14 via an optical system 13a. When the pulsed laser beam is irradiated onto the subject 14, the detection target, such as hemoglobin inside the subject, absorbs the energy of the laser beam, and the temperature of the detection target increases according to the absorbed energy amount. This causes an instantaneous expansion of the detection target, and an photoacoustic wave (elastic wave) is generated. The generated photoacoustic wave is received by the photoacoustic transducer array 4b, and is processed for image reconstruction by the photoacoustic signal processing unit (second signal processing unit) 9 via the receive amplifier 7 and A/D converter 8. The reconstructed photoacoustic signal is stored in the image memory in the image processing unit 11 as a brightness signal or the like.

In the memory of the image processing unit 11, image data of the three-dimensional image (photoacoustic image) and the tomographic image (ultrasonic image) obtained from the photoacoustic signal processing unit 9 and the ultrasonic signal processing unit 10 are stored. In the image processing unit 11, a composite image, combining the photoacoustic analysis image, such as blood vessels, and a tissue image using the ultrasonic echo, is created based on this image data and angle data of the ultrasonic scanning surface 21 from the system control unit 1, and this combined image is displayed on the image display 12. This display may be a composite image where the tomographic image generated by the ultrasonic echo method is superposed on the three-dimensional image generated by the photoacoustic method, or may be a composite image where the ultrasonic image is superposed on the two-dimensional cross-sectional image and two-dimensional projected image generated by the photoacoustic method. These images may be displayed individually.

### [Probe Configuration]

FIG. 2 shows a probe configuration for simultaneously obtaining the photoacoustic signal and ultrasonic echo signal. FIG. 2A shows an external view of the probe, and FIG. 2B is an enlarged view of the transducer portion. FIG. 2C shows a general configuration of the probe and an image capturing region by the photoacoustic method and the ultrasonic echo method.

As FIG. 2A shows, the probe 100 is comprised of a case 30, cable 31 and transducer portion 4. The transducer portion 4 is comprised of the ultrasonic transducer array 4a and the photoacoustic transducer array 4b. As FIG. 2B shows, the photoacoustic transducer array 4b is a two-dimensional array, around which a light irradiation aperture 23, for the pulsed laser light to enter, is disposed. The ultrasonic transducer array 4a has an arrayed (linear) structure in which a plurality of one-dimensional transducer columns are arrayed in sequence. Here a number of devices included in one transducer column is much greater than the number of columns. This structure is actually a two-dimensional array, but it can be regarded as an approximate one-dimensional array, and is also called a 1.75-dimensional arrayed transducer. The ultrasonic transducer array 4a may be a true one-dimensional array.

The ultrasonic transducer array 4a and the photoacoustic transducer array 4b are disposed side by side in a direction perpendicular to the linear scanning direction of the ultrasonic transducer array 4a. Since the ultrasonic transducer array 4a has a plurality of transducer columns, the beam can be tilted in a direction perpendicular to the linear scanning direction by the beam forming processing, so that an ultrasonic beam can be transmitted/received diagonally with respect to the subject surface.

A matching layer, backing and wiring are disposed respectively on the top face and bottom face of the transducer array, and an acoustic lens is disposed on the top face of the ultrasonic transducer array, but these are omitted in the illustration.

The general configuration of the probe will now be described with reference to FIG. 2C. In the probe 100 according to this embodiment, the ultrasonic transducer array 4a, photoacoustic transducer array 4b, light entrance prisms 16a and 16b, and optical transmission path 17 are formed on a protective plate 15. In the optical transmission path 17, a semitransparent mirror film 18 and a total reflection mirror film 19 are formed. The pulsed laser beam generated by the light source 13 propagates through the optical transmission path 17, and a part or preferably half of fluence thereof is reflected by the semitransparent mirror film 18, transmitted through the protective film 15 by the light entrance prism 16a, and is irradiated onto the subject 14. The pulsed laser beam transmitted through the semitransparent mirror film 18 in the optical transmission path 17 is reflected by the total reflection film 19, is transmitted through the protective plate 15 by the light entrance prism 16b, and is irradiated onto the subject 14. The optical transmission path 17 can be anything if the pulsed laser beam can be transmitted through without loss, and can be created by an optical fiber bundle or glass block material. If glass block material is used, the semitransparent mirror film 18 and the total reflection mirror film 19 can be formed by multilayer thin films matching the wavelength of the pulsed laser beam on a block laminating surface or end surface. The configuration inside the optical transmission path 17 functions to spatially propagate the pulsed laser beam, and the semitransparent mirror film 18 and the total reflection mirror film 19 may be constructed using a semitransparent mirror and a total reflection mirror respectively. In this case, the optical transmission path 17 is enclosed with a lens barrel so as to be separated from the outside. The light entrance prisms 16a and 16b may also be constituted by glass block material, but an equivalent effect can be obtained even if a total reflection mirror is used instead. If the optical transmission path 17 is constituted by an optical fiber bundle, the pulsed laser beam may be irradiated onto the subject 14 directly from the optical transmission path 17 using the plasticity of the optical fiber bundle.

In this embodiment, the pulsed laser beam for irradiation is irradiated onto the subject 14 via the light irradiation aperture 23 around the photoacoustic transducer array 4b. The pulsed laser beam diagonally enters the subject 14 by the light entrance prisms 16a and 16b, so as to cross directly under the photoacoustic transducer array 4b. The portion where the pulsed laser beam is irradiated (front portion of the photoacoustic transducer array 4b) is a photoacoustic image capturing region 20 where a three-dimensional image is captured by the photoacoustic spectral method. An advantage of this configuration is that the photoacoustic image capturing region 20 under the photoacoustic transducer array 4b can be irradiated with an approximate uniform fluence.

If the subject 14 is thin, the pulsed laser may be irradiated onto the subject 14 from the opposite side of the photoacoustic transducer array 4b. The subject 14 may be irradiated from both the photoacoustic transducer array 4b side and the opposite side thereof, then the light irradiation intensity in the thickness direction of the subject 14 can be uniform. If the subject 14 is thick, however, it is difficult for the pulsed laser beam to transmit through the subject 14, so it is preferable that the pulsed laser beam enters at least from the photoacoustic transducer array 4b side, like this configuration.

The ultrasonic transducer array 4a transmits the ultrasonic beam, and receives the reflected waves of this beam in the subject 14 as an ultrasonic echo signal. By performing beam forming processing on the received ultrasonic waves, directivity can be provided to the received beam. The ultrasonic transmission/reception beam is scanned in the scanning direction (direction perpendicular to the page face in FIG. 2C). Thereby the tomographic image of the subject 14 is obtained on the ultrasonic scanning surface 21. In other words, the ultrasonic scanning surface 21 is an image capturing region (image capturing cross-section) by the ultrasonic echo method. It is preferable to dispose an ultrasonic standoff 29 for irradiating the ultrasonic beam onto the subject 14 in a tilted state with respect to the surface of the subject, to improve tomographic image capturing.

### [Operation of Ultrasonic Transducer Array]

As mentioned above, according to this embodiment, the direction of the ultrasonic beam which is transmitted to/received from the ultrasonic transducer array 4a, can be tilted in a direction perpendicular to the scanning direction (horizontal direction in FIG. 2C) by the transmission/reception beam former. For this, the ultrasonic transducer array 4a has a plurality of transducer columns. Now the beam steering processing for tilting the beam direction will be described.

As mentioned above, a group of transducers are disposed in a matrix in the ultrasonic transducer array 4a. For explanatory purposes, the ultrasonic beam scanning direction (in a direction perpendicular to the page face in FIG. 2C, which corresponds to the “first direction” of the present invention) is called a “lateral direction”, and a direction perpendicular to this direction (horizontal direction in FIG. 2C, which corresponds to the “second direction” of the present invention) is called an “elevation direction”. The ultrasonic beam scanning is performed by moving the ultrasonic aperture in the lateral direction. The transmission beam former 2 and the reception beam former 6 perform beam scanning by selecting transducers constituting the ultrasonic aperture.

At this time, transmission signals, providing a different delay time to each transducer arrayed in the elevation direction, is input, whereby the transmission beam is steered to the direction to a device of which delay time is relatively lower. The tilt amount of steering, that is the transmission/reception direction of the ultrasonic beam on a plane perpendicular to the linear scanning direction is controlled by the relative amount of the delay time. The user can specify this tilt amount of the steering via an input unit, which is not illustrated. Therefore the user can adjust the inclination of the tomographic image acquisition surface to be a desired angle while checking the obtained image.

In the same manner, by providing a different delay time to each transducer arrayed in the elevation direction when outputting the receive signals from the transducers, the reception beam can be steered to a direction of a device of which delay time is relatively lower in the phasing addition. The tilt amount of the steering is controlled by the relative delay time. Then as is well known in this technical field, the transmission/reception beam is focused by providing a delay to the signal of each transducers in the ultrasonic aperture.

By scanning the ultrasonic beam tilted in the elevation direction in the lateral direction like this, the inclination of the ultrasonic scanning surface 21 is controlled, and the image capturing cross-section by the ultrasonic echo method intersecting with the photoacoustic image capturing region 20 can be changed.

The ultrasonic transducer array 4a according to this embodiment has the characteristics of the above operation. The above mentioned phrase “1.75-dimensional array” is an expression that refers not only to the shape of a transducer array, but also includes the drive method thereof. In other words, the transducer array of which numbers of devices in horizontal and vertical lines are the same or are nearly the same may be used for the ultrasonic transducer array. In this case, the angle formed by the normal line on the transmission/reception surface of the transducers and the ultrasonic beam may be maintained at a specified angle, so that the ultrasonic aperture portion shifted one-dimensionally for a linear scan in order to create a tomographic image at this angle. Normally it is desirable that a number of devices in the scanning direction is high in order to take a wide screen width, but a number of devices for tilting (steering) the ultrasonic beam can be less than this. Therefore in terms of cost, it is desirable to use a transducer array of which horizontal and vertical device arrays are different in shape.

### [Operation of Photoacoustic Transducer Array]

The photoacoustic transducer array 4b is a transducer group disposed in a two-dimensional array form. Unlike ultrasonic transducer array 4a, device selection for creating the aperture portion for receiving photoacoustic waves and scanning of the beam by moving the aperture portion are not performed. The photoacoustic transducer array 4b utilizes the receive signals from approximately all the devices all the time during reception for constructing a three-dimensional image. Ultrasonic waves are not transmitted. The photoacoustic waves from a desired three-dimensional image capturing region, which are generated by light irradiation, are received approximately at the same time by each device of the photoacoustic transducer array 4b, except for the difference of the propagation time thereof, and a three-dimensional image is constructed using all the photoacoustic signals received by each device. For this, photoacoustic signals are obtained instantaneously.

In the present invention, the photoacoustic transducer array 4b, which can acquire signals for a three-dimensional image all at once and the ultrasonic transducer array 4a which requires transmission/reception beam scanning for creating a tomographic image, are provided separately. Furthermore, in order to combine both of these captured images appropriately, the angle of the ultrasonic scanning surface created by the ultrasonic transducer array 4a can be controlled.

### [Characteristics of Transducer]

The ultrasonic transducer array 4a and the photoacoustic transducer array 4b have the following characteristic differences in addition to the above mentioned operational differences.

The ultrasonic transducer array 4a, which is used for drawing the shape information inside the subject, is comprised of transducers which can transmit/receive ultrasonic waves having a higher frequency than the photoacoustic transducer which obtains function information. Here the frequency band of the ultrasonic transducer array 4a is typically about 7 to 12 MHz. The shape information is information based on the shape inside the subject, and refers to information which is obtained by a normal ultrasonic pulse echo method. Also in the case of the ultrasonic transducer array 4a, the transducers must simultaneously satisfy the transmission/reception characteristics so as to transmit/receive ultrasonic waves. For example, devices which have high SNR for reception and devices which have resistance to high voltage to be applied upon transmission are required, and the selection of transducers is limited by these requirements.

On the other hand, the photoacoustic transducer array 4b, which is used for drawing function information inside the subject, is comprised of transducers which can receive ultrasonic waves (photoacoustic waves) having a lower frequency than the ultrasonic transducers which obtain shape information. Here the frequency band of the photoacoustic transducer array 4b is typically 1 to 4 MHz. The function information refers to information obtained by the photoacoustic spectral analysis method (photoacoustic imaging method), and is information on density of a specific substance in the subject, such as glucose and hemoglobin. Although high SNR is demanded for the photoacoustic signal in order to obtain this function information, dedicated transducers for high SNR upon reception can be selected since they are separate from ultrasonic transducers which transmit/receive as described in this embodiment, which is an advantage.

For example, a piezoelectric device which performs mutual conversion between electric signals and mechanical vibration (ultrasonic waves) is used as the transducer constituting the ultrasonic transducer array 4a. On the other hand, any detector can be used as the transducer constituting the photoacoustic transducer array 4b only if acoustic waves can be detected. For example, a transducer using piezoelectric phenomena, a transducer using resonance of light, and a transducer using change of capacity, can be used. Of these, a transducer having high receive SNR can be used according to the intended use. For example, to receive acoustic waves generated from various sized detection targets, a transducer using change of capacitance of which detection frequency band is wide, or a plurality of transducers having different detection bands, may be used.

### [How to Fabricate Probe]

The probe 100 according to this embodiment can be fabricated as follows, for example. First the ultrasonic transducer array 4a (one-dimensional array transducers) and the photoacoustic transducer array 4b (two-dimensional array transducers) are fabricated in a conventional method. This is implemented by extracting the piezoelectric vibrator, securing it to a backing material, dicing the vibrator, gluing it to the acoustic matching layer, and routing the wiring unit. In the ultrasonic transducer, an acoustic lens is installed. The ultrasonic transducer array 4a and the photoacoustic transducer array 4b are aligned with spacing, and are secured by molding. And finally this unit is inserted into a housing.

The fabricated ultrasonic transducer array 4a (one-dimensional array transducers) and the photoacoustic transducer array 4b (two-dimensional array transducers), which are created separately, may be disposed in parallel.

### [Advantage of This Embodiment]

According to this embodiment, the three-dimensional image generated by the photoacoustic method and the two-dimensional tomographic image generated by the ultrasonic echo method can be simultaneously obtained, so the position of a specific structure in the tissue, included in the functional image generated by the photoacoustic method, can be confirmed in the tomographic image generated by the ultrasonic echo method, where the structure of the entire tissue can be obtained.

Since the transducer array for the photoacoustic method and the transducer array for the ultrasonic echo method are independently disposed, devices matching the conditions can be used. Therefore both the images of the photoacoustic method and the images of the ultrasonic echo method can be captured under good conditions, and good images can be obtained.

Furthermore the image capturing region of the photoacoustic method and the image capturing region of the ultrasonic echo method overlap, so both of the images which are simultaneously captured and combined can be displayed in real-time. Even if the images are captured in time-division in order to avoid interference of signals, an image of a same area can be captured almost at the same timing. As to the conventional devices whose image capturing regions thereof are different, in order to obtain images of a same area by both methods, the probe must be moved and images must be recaptured, that is, information at the same timing cannot be obtained.

The angle of the ultrasonic tomographic image (inclination of the ultrasonic beam) can be controlled, so the user can select a cross-section of the tissue structure to be the reference. Since the tomographic image can be displayed by selecting a cross-section where the characteristic shape in the subject can be extracted, a good region can be specified when the photoacoustic analysis region is set. Furthermore the cross-section of the ultrasonic image can be changed when the photoacoustic analytical characteristic in the subject and the tissue structure based on the ultrasonic echo are observed, therefore the photoacoustic analytical characteristic and the tissue structure can be compared in a cross-section in a wide range.

### Second Embodiment

In the first embodiment, the ultrasonic transducer array 4a has a plurality of transducer columns, and the inclination of the ultrasonic scanning surface is controlled by providing a delay time among devices in the elevation direction. In this embodiment, the ultrasonic transducer array 4a is mechanically tilted.

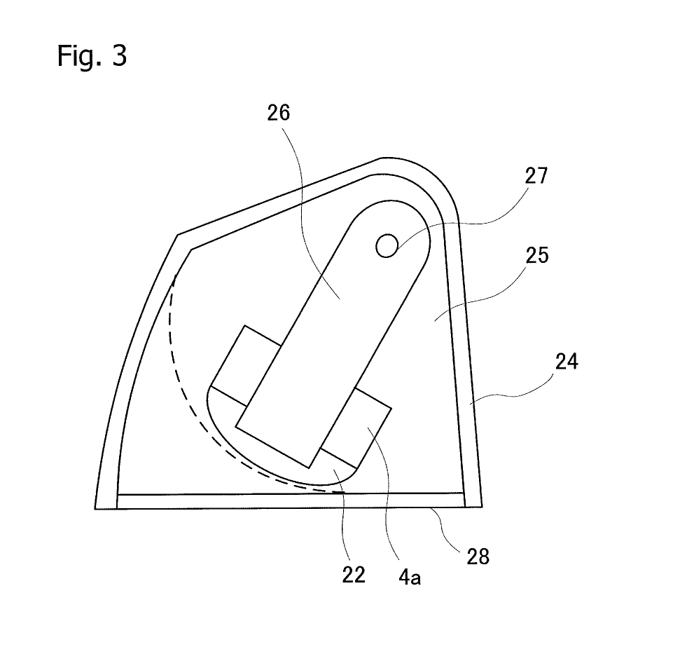

FIG. 3 shows a configuration for tilting the ultrasonic transducer array according to this embodiment. The ultrasonic transducer array 4a is supported by a support arm 26, and the support arm 26 can rotate as specified by a rotation motor, which is not illustrated, via a rotation axis 27. A rotation sensor is attached to the rotation motor, so as to measure the rotation angle of the rotation axis 27. The rotation motor and the rotation sensor are connected to the system control unit 1, and as soon as the rotation motor is driven by a drive signal from the system control unit 1, a tilt angle information signal of the support arm 26 is transmitted to the system control unit 1 by the rotation sensor. The system control unit 1 detects a tilt angle of the support arm 26 based on the tilt angle information signal, and drives the rotation motor by a drive signal, so as to set the tilt angle of the support arm 26 at a desired angle. The support arm 26, rotation axis 27, rotation sensor and rotation motor correspond to the rotation mechanism according to the present invention.

By the above operation, the inclination of the ultrasonic transducer array 4a in the elevation direction can be set at a desired angle. The ultrasonic transducer array 4a, support arm 26 and rotation axis 27 are stored in a packaging material 24 filled with oil 25 for propagating the ultrasonic waves without attenuation. It is preferable that an ultrasonic matching layer 28 is formed on a surface of the packaging material 24 contacting the subject 14, so that the ultrasonic waves transmit without being reflected.

In this configuration, the angle of the ultrasonic beam can be changed, and the cross-angle of the ultrasonic scanning surface 21 and the photoacoustic imaging region 20 can be changed by controlling the inclination of the ultrasonic transducer array 4a itself, so beam forming processing for steering is unnecessary. Therefore the transducers of the transducer array 4a can be a one-dimensionally structured column, and a number of transducers and signal lines thereof can be decreased. Since ultrasonic beam steering is unnecessary in the transmission beam former 2 and reception beam former 6, the circuit configuration scale of each beam former can be smaller than the above mentioned embodiment, which is an advantage. However if the transducers of the transducer array 4a is constructed to be one column, it is preferable to dispose the acoustic lens 22 on the transmission/reception surface of the transducer array 4a, and focus the ultrasonic beam in the elevation direction.

In order to obtain a desired cross-sectional scanning angle, the mechanical tilt control of the transducer array surface and beam steering by the signal delay control among devices may be combined.

### EXAMPLE 1

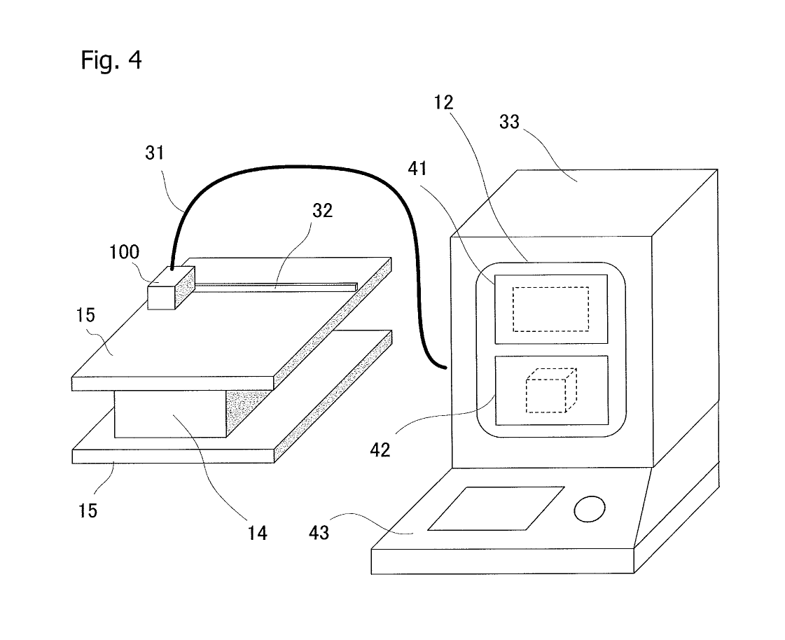

An example of a three-dimensional photoacoustic imaging apparatus according to the present invention will now be described with reference to FIG. 4. A subject 14 is interposed between two protective plates 15. A probe 100 and a guide support (guide unit) 32 are disposed on the protective plate 15. The probe moves along the guide support 32 by a stepping line motor (shifter), which is not illustrated. The probe 100 is connected to a main unit 33 via a cable 31. The stepping line motor is driven by a drive signal from a system control unit 1 in the main unit 33. As shown in FIG. 2C, the main unit 33 has the system control unit 1, transmission amplifier 3, transmission beam former 2, receive amplifiers 5 and 7, reception beam former 6, A/D 8, photoacoustic signal processor 9, ultrasonic signal processor 10, image processor 11, image display 12 and other components. The main unit 33 also has a console 43 used for operation input.

A pulsed laser light source 13 may be disposed in the probe 100, or may be disposed outside, so that the generated laser beam is guided to the probe 100 via a transmission line, which is not illustrated. The ultrasonic transducer array 4a inside the probe 100 is disposed so that the lateral direction thereof is perpendicular to the direction along the guide support 32, and the elevation direction thereof is parallel with the direction along the guide support 32. Hence the movement of the probe is controlled in the elevation direction by the guide support 32. The image display unit 12 houses a photoacoustic image display unit 42 for displaying a photoacoustic analytical image and an ultrasonic image display unit 41 for displaying a tomographic image of the tissue generated by the ultrasonic echo method.

The ultrasonic beam is transmitted/received by the ultrasonic transducer array 4a in the probe 100 while scanning the ultrasonic beam in the lateral direction, and the tomographic image of the tissue is generated by the ultrasonic echo in the main unit 33, as mentioned above, and is displayed on the ultrasonic image display 41 in the image display 12 in real-time. It is preferable that the user adjusts the angle of the ultrasonic scanning surface 21 via the console 43 according to the thickness of the subject 14, so that the tomographic surface of the ultrasonic tomography includes the entire width of the photoacoustic imaging region 20. The position of the image capturing cross-section can be adjusted with reference to this ultrasonic tomographic image displayed in real-time.

Then the pulsed laser light source is driven by a drive signal from the system control unit 1, a pulsed laser light is irradiated from a light aperture in the probe 100 to the subject 14, and photoacoustic signals are obtained by the photoacoustic transducer array 4b at the same time. Photoacoustic analytical image data is generated using the obtained photoacoustic signals in the main unit 33 according to the procedure described above, and a three-dimensional photoacoustic image is generated by the image processor 11, and is displayed on the photoacoustic image display 42 in the image display 12. The image displayed on the photoacoustic image display 42 can be a composite image where a tomographic image generated by the ultrasonic echo method is superposed on a three-dimensional photoacoustic image, or a photoacoustic two-dimensional cross-sectional image, a two-dimensional projected image, or a composite image where these photoacoustic images are superposed on an ultrasonic image. An image displayed on the photoacoustic image display 42 may be an image connecting the captured images of different areas of the subject 14, obtained by the probe 100 moving along the guide support 32.

This example has the advantage in that a photoacoustic analytical image is obtained in a wide range of a subject 14 by moving the probe 100 in a direction along the guide support 32, and a part or all of the image can be displayed. At this time, if an ultrasonic tomographic image crossing the photoacoustic imaging region 20 is obtained simultaneously and displayed in real-time, the user can move the probe 100 while checking the region in the subject 14 to be imaged with the probe 100. In other words, an imaging position and imaging range of the photoacoustic analysis by the probe 100 can be specified while viewing the ultrasonic image on the ultrasonic image display 41, and the photoacoustic analytic imaging range can be determined referring to the tissue structure in the subject based on the ultrasonic image. Since the angle of the tomographic surface of the ultrasonic image can be adjusted, a cross-section of the tissue structure to be the reference can be selected. Therefore a cross-section, where the characteristic shape can be extracted in the structure of the subject 14, can be selected, and the ultrasonic tomographic image of this cross-section can be displayed, therefore a good region can be specified when a photoacoustic analytical region is set using an ultrasonic image. Furthermore the cross-section of the ultrasonic screen can be changed when the photoacoustic analytical characteristic in the subject 14 and the tissue structure generated by an ultrasonic echo are observed using a composite image, so the photoacoustic analytical characteristic and the tissue structure can be compared in a wide range of the cross-section.

In the above description, the photoacoustic image is a three-dimensional image and the ultrasonic image is a two-dimensional tomographic image, but a three-dimensional image generated by the ultrasonic echo method may be displayed. The three-dimensional image by the ultrasonic echo method can be generated by combining a plurality of ultrasonic tomographic images obtained by the probe 100 moving along the guide support 32 according to the position of the probe 100. In this case as well, the following effect is generated since the photoacoustic imaging region 20 and the ultrasonic scanning surface 21 cross and superpose. In other words, the photoacoustic image and the ultrasonic image are superposed in the moving direction, so the entire apparatus can be created to be compact by decreasing the region where the composite image cannot be displayed due to the deviation of these images with respect to the moving distance of the probe 100, that is, by decreasing the moving distance of the probe 100.

In the above example, the probe 100 moves one-dimensionally, but if the probe 100 is moved two-dimensionally on the subject 14 by combining this linear movement in a raster form, a photoacoustic image in a wider range can be generated.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-38977, filed on Feb. 24, 2010, which is hereby incorporated by reference herein in its entirety.

What is claimed is:

1. A subject information processing apparatus comprising:
a first device array for transmitting/receiving an elastic wave;
a first signal processor for generating a tomographic image from a signal received by the first device array;
a second device array for receiving an elastic wave which is generated by irradiating light onto a subject; and
a second signal processor for generating a three-dimensional image from a signal received by the second device array, wherein
the first device array transmits/receives the elastic wave diagonally with respect to a surface of the subject so that a region in the subject where the tomographic image is obtained and a region in the subject where the three-dimensional image is obtained overlap.

2. The subject information processing apparatus according to claim 1, wherein
a tomographic image on a scanning surface is obtained by scanning the elastic wave transmitted from the first device array, and
the scanning surface overlaps with a region of the subject where the light is irradiated.

3. The subject information processing apparatus according to claim 1, wherein
the first device array comprises a plurality of transducers arrayed at least in a first direction,
the second device array comprises a plurality of transducers which are two-dimensionally arrayed,
the first device array and the second device array are disposed side by side in a second direction perpendicular to the first direction, and
the first device array transmits/receives the elastic wave in a direction tilted toward the second direction.

4. The subject information processing apparatus according to claim 3, wherein
the first device array has a two-dimensional array configuration where a plurality of transducers are also arrayed in the second direction, and
a tilt angle of a transmission/reception direction of the elastic wave toward the second direction can be controlled by providing a different delay time to each of the transducers arrayed in the second direction.

5. The subject information processing apparatus according to claim 3, further comprising:
a rotation mechanism for rotating the first device array with the first direction as a rotation axis, wherein
the tilt angle of the transmission/reception direction of the elastic wave toward the second direction can be controlled by rotating the first device array by the rotation mechanism.

6. The subject information processing apparatus according to claim 3, further comprising:
a probe including at least the first device array and the second device array;
a guide for controlling movement of the probe in the second direction; and
a shifter for shifting the probe along the guide.

7. The subject information processing apparatus according to claim 1, further comprising:
a display for displaying the tomographic image generated by the first signal processor and the three-dimensional image generated by the second signal processor in a superposed manner.

8. A subject information processing method for a subject information processing apparatus, the method comprising:
a tomographic image generation step of generating a tomographic image of the subject by irradiating the elastic wave onto the subject, and receiving the echoed elastic wave reflected from inside the subject; and
a three-dimensional image generation step of generating a three-dimensional image of the subject by irradiating the light onto the subject, and receiving the elastic wave generated inside the subject, wherein
in the tomographic image generation step, the elastic wave is transmitted/received diagonally with respect to a surface of the subject so that a region in the subject where the tomographic image is obtained and a region in the subject where the three-dimensional image is obtained overlap.
